(19) United States
(12) Patent Application Publication
(10) Pub. No.: US 2018/0153505 A1
(43) Pub. Date: Jun. 7, 2018

(54) GUIDED NAVIGATION OF AN ULTRASOUND PROBE

(71) Applicant: Bay Labs, Inc., San Francisco, CA (US)

(72) Inventors: Charles Cadieu, San Francisco, CA (US); Ha Hong, Pleasant Hill, CA (US); Kilian Koepsell, San Francisco, CA (US); Johan Mathe, San Francisco, CA (US); Martin Wojtczyk, Pinole, CA (US)

(21) Appl. No.: 15/831,375

(22) Filed: Dec. 4, 2017

(30) Foreign Application Priority Data
Dec. 7, 2016 (FR) 1662038

(51) Int. Cl. A61B 8/00; A61B 34/20

(52) U.S. Cl. CPC A61B 8/4254; A61B 2034/107; A61B 34/20; A61B 8/461

(57) ABSTRACT

Embodiments of the invention provide for the guided navigation of an ultrasound probe. In an embodiment of the invention, an ultrasound navigation assistance method includes acquiring an image by an ultrasound probe of a target organ of a body. The method also includes processing the image in connection with an estimator such as a neural network. The processing in turn determines a deviation of a contemporaneous pose evident from the acquired image from an optimal pose of the ultrasound probe for imaging the target organ. Finally, the method includes presenting the computed deviation to an end user operator of the ultrasound probe.

This invention was made with government support under SBIR Phase I: Semantic Video Analysis for Video Summarization and Recommendation Proposal Number IIP-1416612 awarded by National Science Foundation (NSF). The United States Government has certain rights in the invention.

### BACKGROUND OF THE INVENTION

### Field of the Invention

The present invention relates to ultrasound imaging and more particularly to ultrasound image acquisition.

### Description of the Related Art

Ultrasound imaging, also known as sonography, is a medical imaging technique that employs high-frequency sound waves to view three-dimensional structures inside the body of a living being. Because ultrasound images are captured in real-time, ultrasound images also show movement of the internal organs of the body as well as blood flowing through the blood vessels of the human body and the stiffness of tissue. Unlike x-ray imaging, ultrasound imaging does not involve ionizing radiation thereby allowing prolonged usage of ultrasound imaging without threatening tissue and internal organ damage from prolonged radiation exposure.

To acquire ultrasound imagery, during an ultrasound exam, a transducer, commonly referred to as a probe, is placed directly on the skin or inside a body opening. A thin layer of gel is applied to the skin so that the ultrasound waves are transmitted from the transducer through the medium of the gel into the body. The ultrasound image is produced based upon a measurement of the reflection of the ultrasound waves off the body structures. The strength of the ultrasound signal, measured as the amplitude of the detected sound wave reflection, and the time taken for the sound wave to travel through the body provide the information necessary to compute an image.

Compared to other prominent methods of medical imaging, ultrasound presents several advantages to the diagnostician and patient. First and foremost, ultrasound imaging provides images in real-time. As well, ultrasound imaging requires equipment that is portable and can be brought to the bedside of the patient. Further, as a practical matter, the ultrasound imaging equipment is substantially lower in cost than other medical imaging equipment, and as noted, does not use harmful ionizing radiation. Even still, the production of quality ultrasound images remains highly dependent upon a skilled operator.

In this regard, depending upon the portion of the body selected for imaging, the skilled operator must know where to initially place the ultrasound probe. Then, the skilled operator must know how to spatially orient the probe and finally, the skilled operator must know where to move the probe so as to acquire the desired imagery. Generally, the ultrasound operator is guided in the initial placement, orientation and movement of the probe based upon the visual feedback provided by the imagery produced during the ultrasound. Thus, essentially, the navigation of the probe is a manual process consisting of iterative trial and error. Plainly, then, the modern process of ultrasound navigation is not optimal.

### BRIEF SUMMARY OF THE INVENTION

Embodiments of the present invention address deficiencies of the art in respect to ultrasound probe navigation and provide a novel and non-obvious method, system and computer program product for the guided navigation of an ultrasound probe. In an embodiment of the invention, an ultrasound navigation assistance method includes acquiring an image by an ultrasound probe of a target organ of a body. The method also includes submitting the image processing in connection with an estimator formed as a function or programmatic approximator, including by way of example, a classifier, regressor, a state machine or a neural network. The processing with respect to the estimator produces as an output a deviation between a contemporaneous pose of the ultrasound probe, namely the position and orientation of the ultrasound probe relative to the target organ, and an optimal pose of the ultrasound probe for imaging the target organ. Finally, the method includes presenting the deviation to an end user operator of the ultrasound probe.

In one aspect of the embodiment, the contemporaneous pose of the ultrasound probe is additionally improved based upon linear and angular movement data received from an inertial measurement system including at least one of an accelerometer, gyroscope and magnetometer. In another aspect of the embodiment, the computed deviation is presented visually in a display of a computer system coupled to the probe, audibly through a varying of a tone based upon a proximity of the probe to the optimal pose, audibly by varying a frequency of repeatedly audibly presenting a short-duration sound based upon a proximity of the probe to the optimal pose, or haptically through a varying of vibrations of the probe based upon a proximity of the probe to the optimal pose.

In another embodiment of the invention, an ultrasound imaging data processing system is configured for ultrasound navigation assistance. The system includes a computer with memory and at least one processor, a display coupled to the computer, beamformer circuitry coupled to the computer and the display, and an ultrasound probe that has an array transducer connected to the beamformer circuitry. The system additionally includes a navigation assistance module executing in the memory of the computer. The module includes program code enabled upon execution by the processor of the computer to acquire an image by the ultrasound probe of a target organ of a body, to submit the image for processing in connection with an estimator, for instance, a neural network, so as to product a deviation between a contemporaneous pose of the ultrasound probe relative to the target organ and an optimal pose of the ultrasound probe for imaging the target organ, and to present the computed deviation to an end user operator of the ultrasound probe.

Additional aspects of the invention will be set forth in part in the description which follows, and in part will be obvious from the description, or may be learned by practice of the invention. The aspects of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE SEVERAL VIEWS OF THE DRAWINGS

The accompanying drawings, which are incorporated in and constitute part of this specification, illustrate embodiments of the invention and together with the description, serve to explain the principles of the invention. The embodiments illustrated herein are presently preferred, it being understood, however, that the invention is not limited to the precise arrangements and instrumentalities shown, wherein:

FIG. 1 is a pictorial illustration of a process for guided navigation of an ultrasound probe

FIG. 2 is a schematic illustration of an ultrasound data processing system configured for guided navigation of an ultrasound probe; and,

FIG. 3 is a flow chart illustrating a process for guided navigation of an ultrasound probe.

### DETAILED DESCRIPTION OF THE INVENTION

Embodiments of the invention provide for guided navigation of an ultrasound probe. In accordance with an embodiment of the invention, an ultrasound probe is placed on the surface of a body. Then, imagery of a target organ of the body is acquired and a deviation of a contemporaneous pose of the ultrasound pose evident from the acquired image from an optimal pose for the target organ is presented to an end user operator of the ultrasound probe. For example, the deviation is presented visually in respect to a corresponding display, audibly by way of an audible guidance signal, or in the alterative, by way of the text to speech presentation of textual instructions, or haptically through the outer shell of the ultrasound probe.

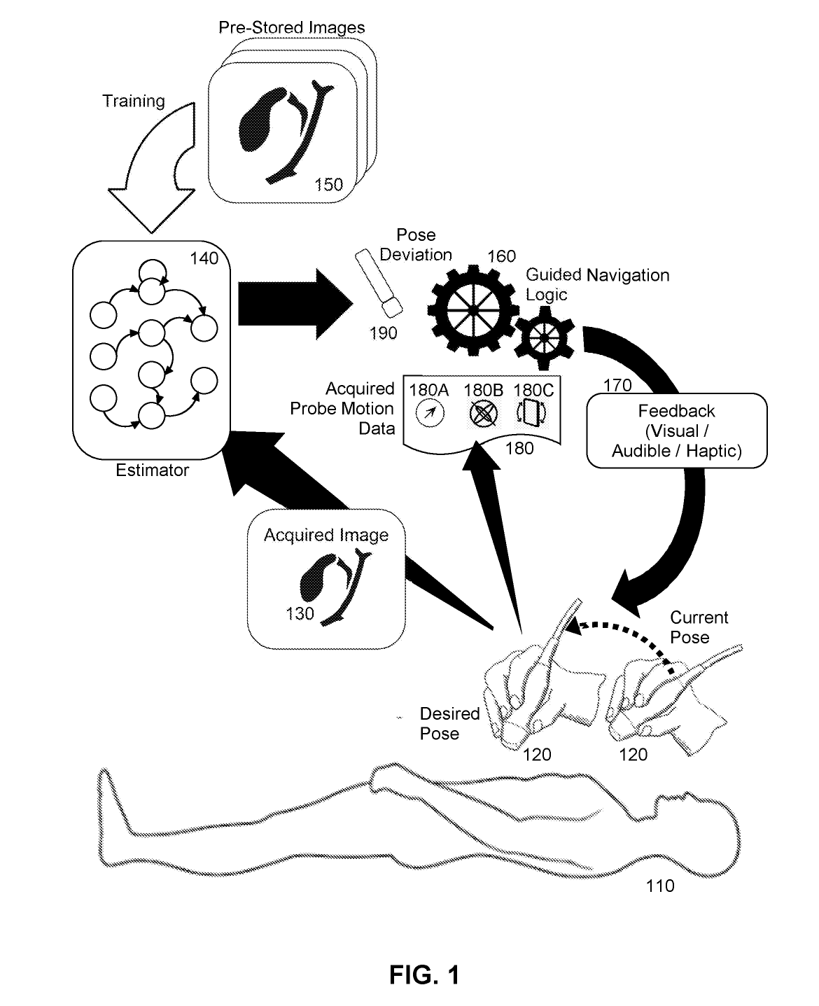

In illustration, FIG. 1 is a pictorial illustration of a process for guided navigation of an ultrasound probe. As shown in FIG. 1, an ultrasound probe 120 is placed upon an outer surface of a body 110 such as a human form. Imagery 130 of a target organ is acquired by the operator of the ultrasound probe 120 and the image 130 of the target organ is presented input to an estimator 140, such as a neural network. The estimator 140 is trained based upon a set of training images 150 of one or more different target organs, each with a known probe pose deviation from the optimal probe pose so that the input of the contemporaneously acquired image 130 to the estimator 140 produces an output of a deviation 190 of the contemporaneous pose of the ultrasound probe 120 from an optimal pose of the ultrasound probe 120.

Optionally, the ultrasound probe 120 acquires probe orientation and movement data 180 including magnetomic information 180A, gyroscopic information 180B and accelerometric information 180C indicating an orientation and movement of the ultrasound probe 120 so as to compute the change in the pose of the ultrasound probe 120. Guided navigation logic 160 then processes the probe orientation and movement data 180 so as to better compute the deviation from the optimal pose 190 in consideration not only of the pose deviation 190 output by the estimator 140 in respect to the acquired image 130 but also in consideration of the change in the pose of the ultrasound probe 120 determined from the probe orientation and movement data 180.

Guided navigation logic 160 then processes the pose deviation 190 of the ultrasound probe 120 and emits feedback 170 in the form of visual feedback such as a three-dimensionally rendered scene with the two probe models showing current and optimal probe poses with suggested maneuver; the amount of agreement between the current and optimal poses; or red, green or yellow colors indicating how large an adjustment of the orientation of the ultrasound probe 120 is required to approach the optimal pose, audible feedback such as a tone, or haptic feedback. In regard to the latter, in one aspect of the invention the ultrasound probe 120 may be caused to vibrate more intensely or with greater frequency responsive to the pose deviation 190.

In respect to the former, in one aspect of the invention the ultrasound probe 120 may be caused to emit a sound that is more intense of a different tone when the ultrasound probe 120 based upon the magnitude of the pose deviation 190. As well the ultrasound probe 120 may be caused to emit a short-duration sound such as a click or pop repeatedly with a frequency related to the magnitude of the pose deviation 190. Alternatively, in another aspect of the invention the ultrasound probe 120 may be caused to vibrate more intensely or with greater frequency when the ultrasound probe 120 is moved in a compliant manner based upon a magnitude of the pose deviation 190.

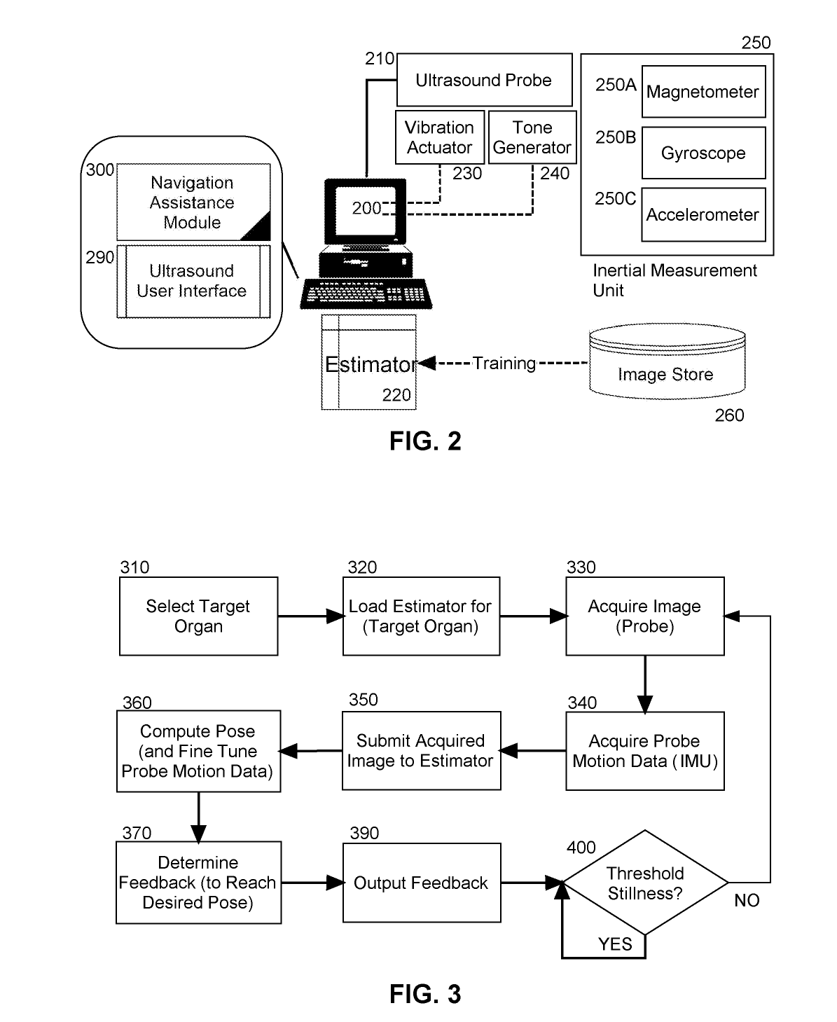

The process described in connection with FIG. 1 may be implemented in an ultrasound data processing system. In further illustration, FIG. 2 schematically illustrates an ultrasound data processing system configured for guided navigation of an ultrasound probe. The system includes an ultrasound probe 210 coupled to a host computing system 200 of one or more computers, each with memory and at least one processor. The ultrasound probe 210 is enabled to acquire ultrasound imagery by way of a transducer connected to beamformer circuitry in the host computing system 200, and transmit the acquired ultrasound imagery to the beamformer circuitry of the host computing system 200 for display in a display of the host computing system 200 through an ultrasound user interface 290 provided in the memory of the host computing system 200.

The ultrasound probe 210 includes an electromechanical vibration generator 230 such as a piezo actuator, and a tone generator 240. The electromechanical vibration generator 230 may be driven in the ultrasound probe 210 to cause the ultrasound probe 210 to vibrate at a specific frequency and for a specific duration as directed by the host computing system 200. As well, the tone generator 240 may be driven in the ultrasound probe 210 to cause the ultrasound probe 210 to emit an audible tone at a specific frequency and amplitude and for a specific duration as directed by the host computing system 200. Optionally, the tone generator 240 may be disposed in the host computing system 200.

An image data store 260 stores therein a multiplicity of different ultrasound images previously acquired in a controlled setting where the pose deviation of each image is known. The images of the image store 260 are provided as training images in training an estimator 220 such as a neural network providing decisioning of a deviation from an optimal probe pose relative to an input image of a target organ of a human form. In this regard, the estimator 220 includes a multiplicity of nodes processing different extracted features of an acquired image so as to decision a pose of the ultrasound probe providing a deviation of the decisioned pose from a known, optimal pose in imaging a target organ. In this regard, the pose can be represented mathematically in Euclidian space or any array of numbers.

Finally, a navigation assistance module 300 is coupled to the ultrasound user interface 290. The navigation assistance module 300 includes program code that when executed in the memory of the host computing system 200 acquires a contemporaneous ultrasound image by the ultrasound probe 210 and processes the acquired ultrasound image in the host computing platform 200 utilizing the estimator 220. The program code of the navigation assistance module 300 during execution in the memory of the host computing system 200 then receives with the assistance of the estimator 220 a computed deviation of a pose evident from the acquired image, from an optimal pose of the ultrasound probe 210.

Optionally, the ultrasound probe 210 includes an inertial measurement unit 250. The inertial measurement unit 250 includes each of a magnetometer 250A, a gyroscope 250B, and an accelerometer 250C. As such, data acquired by the inertial measurement unit 250 is translated in the host computing system 200 to estimate a change in pose for a given time interval, for instance by measuring linear acceleration and angular velocity of the probe 210. This change in pose can be combined with an estimator-derived pose deviation to obtain a more precise pose estimate. One possible example includes the use of a Kalman filter.

For instance, algorithmically, the process utilizing the inertial measurement unit 250 to tune a determined deviation from the estimator 220 can be expressed as follows:

1. Let I(t) be the image acquired by the ultrasound probe 210 at time t.

2. Let f be an estimator such as neural network 220 that from an acquired image I(t) outputs the image-estimated pose p(t)_{img} at time t relative to the optimal pose (i.e., deviation of the pose from the optimal pose). That is, p(t)_{img}=f(I(t)).

3. Between t₀ and t₁ (>t₀), the change of pose Δp(t₁, t₀)_{img} can be computed as: Δp(t₁, t₀)_{img}=p(t₁)_{img}−p(t₀)_{img}. Obviously, p(t₁)_{img}=p(t₀)_{img}+Δp(t₁, t₀)_{img}.

4. The change of pose between to and t₁is measured from the inertial measurement unit 250 and denoted as Δp(t₁, t₀)_{IMU}. Δp(t₁, t₀)_{img} and Δp(t₁, t₀)_{IMU} are combined to produce a better estimate of the pose change by using a Kalman filter expressed as Δp(t₁, t₀)_{K}=K(Δp(t₁, t₀)_{img}, Δp(t₁, t₀)_{IMU}; p(t₀)_{img}) where K is a Kalman filter (that takes image-based pose change, inertial measurement unit 250 based pose change, and the pose at to as inputs), Δp(t₁, t₀)_{K} is the combined pose change that is expected to be more accurate than either Δp(t₁, t₀)_{img} or Δp(t₁, t₀)_{IMU} alone.

5. With the more accurate Δp(t₁, t₀)_{K}, it is then possible to estimate more accurate absolute pose p(t₁)_{K} at time p(t₁)_{K}=p(t₀)_{img}+Δp(t₁, t₀)_{K}

6. For all subsequent time points: p(tⱼ₊1)_{K}=p(tⱼ)_{K}+Δp(tⱼ, tⱼ+1)_{K}

In any event, based upon the computed deviation, the program code of the navigation assistance module 300 then determines corresponding feedback to be presented through the ultrasound probe 210. For example, the program code of the navigation assistance module 300 may direct the tone generator 240 to emit a particular tone pattern of specific periodicity proportional or inversely proportional to a determined proximity of the ultrasound probe 210 to the optimal pose. As another example, the program code of the navigation assistance module 300 may direct the electromechanical vibration generator 230 of the ultrasound probe 210 to emit a particular vibration of specific intensity proportional or inversely proportional to a determined proximity of the ultrasound probe 210 to the optimal pose.

In yet further illustration of the operation of the navigation assistance module 300, FIG. 3 is a flow chart depicting a process for guided navigation of an ultrasound probe. Beginning in block 310, a target organ within the body is selected in a user interface to an ultrasound application visualizing ultrasound imagery acquired by the ultrasound probe. Subsequently, in block 320 an estimator such as a neural network pertaining to the target organ is loaded into memory of a computing system coupled to the ultrasound probe. In block 330, a contemporaneous ultrasound imagery is acquired by the ultrasound probe and in block 340, optionally, probe orientation and movement data is received from an inertial measurement unit of the ultrasound probe is acquired. In block 350, the contemporaneous ultrasound imagery is processed in connection with to the estimator in the computing system.

In block 360, a deviation of a pose of the ultrasound probe from an optimal pose that is evident from the acquired image is determined based upon the application of the estimator to the acquired image. Optionally, the probe orientation and movement data are used to further improve the accuracy of the determined pose deviation. In block 370, corresponding feedback based upon the deviation is determined such as a graphical representation of the deviation, a particular strength of vibration as part of haptic feedback, or a particular tone of particular frequency, periodicity, amplitude or any combination thereof as part of audible feedback. In block 390, then, the determined feedback is output by the computing system or the coupled ultrasound probe. Finally, in decision block 400, the inertial measurement unit of the ultrasound probe indicates whether or not a threshold change in position or orientation has occurred with respect to the ultrasound probe. If so, or in case where an inertial measurement unit is not present or active, the process may then repeat through block 340.

The present invention may be embodied within a system, a method, a computer program product or any combination thereof. The computer program product may include a computer readable storage medium or media having computer readable program instructions thereon for causing a processor to carry out aspects of the present invention. The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing.

Computer readable program instructions described herein can be downloaded to respective computing/processing devices from a computer readable storage medium or to an external computer or external storage device via a network. The computer readable program instructions may execute entirely on the user's computer, partly on the user's computer, as a stand-alone software package, partly on the user's computer and partly on a remote computer or entirely on the remote computer or server. Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Finally, the terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

Having thus described the invention of the present application in detail and by reference to embodiments thereof, it will be apparent that modifications and variations are possible without departing from the scope of the invention defined in the appended claims as follows:

We claim:

1. An ultrasound navigation assistance method comprising:
acquiring an image by an ultrasound probe of a target organ of a body;
processing the image in connection with an estimator, the estimator producing a deviation of a contemporaneous pose evident from the image from an optimal pose of the ultrasound probe for imaging the target organ; and,
presenting the computed deviation to an end user operator of the ultrasound probe.

2. The method of claim 1, wherein the estimator comprises a neural network.

3. The method of claim 1, wherein the contemporaneous pose is additionally determined based upon probe orientation and movement data received from an inertial measurement system comprising at least one of an accelerometer, gyroscope and magnetometer.

4. The method of claim 1, wherein the computed deviation is presented visually in a display of a computer system coupled to the probe.

5. The method of claim 1, wherein the computed deviation is presented audibly through a varying of a tone based upon a proximity of the probe to the optimal pose.

6. The method of claim 1, wherein the computed deviation is presented audibly by varying a frequency of repeatedly audibly presenting a short-duration sound based upon a proximity of the probe to the optimal pose.

7. The method of claim 1, wherein the computed deviation is presented haptically through a varying of vibrations of the probe based upon a proximity of the probe to the optimal pose.

8. An ultrasound imaging data processing system configured for ultrasound navigation assistance, the system comprising:
a computer with memory and at least one processor;
a display coupled to the computer;
beamformer circuitry coupled to the computer and the display;
an ultrasound probe comprising a transducer connected to the beamformer circuitry; and,
a navigation assistance module executing in the memory of the computer, the module comprising program code enabled upon execution by the processor of the computer to acquire an image by the ultrasound probe of a target organ of a body, to process with an estimator the acquired image by determining a deviation between a contemporaneous pose of the ultrasound probe relative to the target organ evident from the acquired image, and an optimal pose of the ultrasound probe for imaging the target organ, and to present the computed deviation to an end user operator of the ultrasound probe.

9. The system of claim 8, wherein the estimator is a neural network.

10. The system of claim 8, wherein the contemporaneous pose is additionally determined based upon probe orientation and movement data received from an inertial measurement system comprising at least one of an accelerometer, gyroscope and magnetometer.

11. The system of claim 8, wherein the computed deviation is presented visually in a display of a computer system coupled to the probe.

12. The system of claim 8, wherein the computed deviation is presented audibly through a varying of a tone based upon a proximity of the probe to the optimal pose.

13. The system of claim 8, wherein the computed deviation is presented haptically through a varying of vibrations of the probe based upon a proximity of the probe to the optimal pose.

14. A computer program product for ultrasound navigation assistance, the computer program product comprising a computer readable storage medium having program instructions embodied therewith, the program instructions executable by a device to cause the device to perform a method comprising:
acquiring an image by an ultrasound probe of a target organ of a body;
processing the image in connection with an estimator, the estimator producing a deviation of a contemporaneous pose of the ultrasound probe evident from the acquired image from an optimal pose of the ultrasound probe for imaging the target organ; and,
presenting the computed deviation to an end user operator of the ultrasound probe.

15. The computer program product of claim 14, wherein the estimator is a neural network.

16. The computer program product of claim 14, wherein the pose is additionally determined based upon probe orientation and movement data received from an inertial measurement system comprising at least one of an accelerometer, gyroscope and magnetometer.

17. The computer program product of claim 14, wherein the computed deviation is presented visually in a display of a computer system coupled to the probe.

18. The computer program product of claim 14, wherein the computed deviation is presented audibly through a varying of a tone based upon a proximity of the probe to the optimal pose.

19. The computer program product of claim 14, wherein the computed deviation is presented audibly by varying a frequency of repeatedly audibly presenting a short-duration sound based upon a proximity of the probe to the optimal pose.

20. The computer program product of claim 14, wherein the computed deviation is presented haptically through a varying of vibrations of the probe based upon a proximity of the probe to the optimal pose.
